(19) United States
(12) Patent Application Publication
(10) Pub. No.: US 2014/0257039 A1
(43) Pub. Date: Sep. 11, 2014

(54) SURGICAL RETRACTOR WITH SMOKE EVACUATOR

(71) Applicant: Joel Feldman, Cambridge, MA (US)

(72) Inventor: Joel Feldman, Cambridge, MA (US)

(21) Appl. No.: 13/791,139

(22) Filed: Mar. 8, 2013

(51) Int. Cl. A61M 1/00; A61B 1/32

(52) U.S. Cl. CPC A61M 1/008; A61B 1/32; A61M 1/0066
USPC 600/205

(57) ABSTRACT

Disclosed herein are systems, devices, and methods for retracting tissue and removing surgical smoke using a smoke evacuation conduit positioned on or within an internal surface of the retractor blade such that the conduit does not protrude onto the external surface of the blade, along with a smoke intake port on or flush with the exterior surface of the blade and set back from the tip to minimize tissue aspiration. The systems and devices allow for the channeling of surgical smoke away from the surgical site while avoiding occlusion of the surgeon's field of view and preserving the surgeon's freedom of motion.

The present disclosure relates generally to surgical systems, devices, and methods for tissue retraction and the removal of surgical smoke.

Various injuries and surgical incisions necessitate the retraction of tissue and the generation of surgical smoke. For example, tissue is often cauterized during surgical procedures to prevent excessive bleeding. The cautery systems currently in use can generate significant amounts of smoke, which can be unhealthy and unpleasant to the surgical staff and can impair a surgeon's view of a surgical field. Other procedures, such as surgical laser treatments, can also result in the vaporization of tissue and lead to the generation of smoke. To address this problem, various smoke evacuation systems have been developed, including free-standing vacuum-suction systems, systems attached to a cautery device, and systems attached to a tissue retractor. These devices generally require the delivery of negative pressure (i.e., suction) through a cannula. However, the suction sources, including tubes or other suction devices, can obscure the surgeon's view and can interfere with the surgeon's freedom of movement, especially during procedures that have a small field of view. These devices can also suffer from inefficient smoke removal.

With increasing frequency, various kinds of head and neck, abdominal, thoracic, breast, and extremity surgery are performed through relatively small access incisions in the skin. In that regard, there is often a need during surgery to lift soft tissues along and beneath the edge of an incision with a thin-bladed retractor to provide the surgeon with an adequate degree of visualization and maneuverability. At the same time, the surgeon may also cut, remove, or shape the underlying subcutaneous tissues, e.g., with a high-frequency electrosurgical “pencil” or a laser beam wand that simultaneously inhibits bleeding. These hemostatic-dissecting devices, however, can also generate an amount of smoke, the so-called smoke “plume”, which can be unhealthy and unpleasant for the surgical staff to inhale, and may cloud the surgeon's view of the operative field.

To address this problem, various smoke evacuation devices, which connect through flexible tubing to a source of negative pressure, have been deployed. For example, various oral surgical tools, proctoscopes, vaginal speculae, and other devices have been developed for removing smoke while retracting tissue, but such devices are not intended for use with small incision surgeries or position the smoke evacuation devices within the surgeon's field of view. See, e.g., U.S. Patent Publication No. 2012/0015317; Salvati et al., Am. J. Surg. 132: 583-586 (1976); the WELCH ALLYN KLEENSPEC® vaginal specula. The simplest of these devices are rigid or semi-rigid tubes (e.g., a Yankauer or Poole suction instrument), which were originally designed for the removal of body fluids and tissue debris from the operative site, but can also function as a scavenger of surgical smoke. When used for that purpose during a small incision procedure, these devices have some shortcomings. They can require the use of an additional hand to hold them in position, and they occupy space in an already crowded surgical field.

To avoid those handicaps, a number of space-saving instruments have been designed that combine a narrow caliber smoke evacuation cannula (or conduit) with one of the three other instruments usually present in the incision when the “cautery” is being used—the electrosurgical “pencil” itself, a tissue-grasping forceps, and the wound-edge retractor. The first two of these hybrid instruments, however, generally perform poorly as smoke collectors and are cumbersome due to the need to drag the suction tubing as the devices are used. And while the smoke exhaust catheter is thin, it and its means of attachment to the cautery or the forceps can nonetheless create a visual obstruction.

Accordingly, disclosed herein are devices, systems, and methods for the removal of surgical smoke using a smoke evacuation conduit on the interior surface of a retractor blade, where it is hidden from the surgeon's view during use, and a smoke intake port located flush with the exterior surface of the retractor blade, where it avoids impediment of the visual field. In some embodiments, the device comprises a tissue retractor capable of efficiently removing noxious surgical smoke, while avoiding interference with a surgeon's field of view. The surgical smoke can be removed by positioning a smoke intake port about ½ to about 1 inch from the distal tip of the retractor blade, with the intake port connected to a smoke evacuation conduit that delivers negative pressure to the intake port and runs along or partially within the interior surface of the retractor blade and therefore does not obstruct the surgeon's view during use. The positioning of the smoke intake port near but not at the distal tip of the retractor blade optimizes the removal of surgical smoke, while avoiding the aspiration of tissue from the operating site. The retractor and/or evacuation conduit can be disposable, and can be permanently attached or can be separable (e.g., using snaps to attach a disposable evacuation conduit to a reusable, autoclavable retractor blade).

In some embodiments, the device is a surgical instrument for simultaneously retracting tissues and evacuating surgical smoke through a source of suction. The conduit can deliver negative pressure to a surgical site through a smoke intake port positioned near the distal end of the retractor blade. The devices and systems are capable of providing enhanced smoke removal while avoiding obstruction of the surgeon's view of the surgical field and/or without interfering with freedom of motion. In some embodiments, the device comprises a narrow-caliber smoke evacuation conduit positioned on the interior surface of the retractor blade. In certain embodiments, a smoke intake port is positioned flush with the exterior surface of the retractor blade and removes the surgical smoke by pulling the smoke through a channel in the retractor blade and into the evacuation conduit positioned on the interior surface of the retractor (i.e., the surface facing away from the surgeon's field of view). In some embodiments, the smoke intake port is positioned behind (i.e. proximal to) the distal end of the retractor blade such that it is near the center of the surgical smoke plume where it can effectively gather smoke while reducing or preventing the aspiration of tissue from the surgical site.

In some embodiments, the device is a medical instrument that comprises a small hole that is flush with an exterior convex surface of the instrument's retractor blade and provides a smoke intake port. In certain embodiments, the smoke intake port is positioned about 1 inch from the distal end of the retractor blade where it can collect surgical smoke without aspirating surrounding tissues. The smoke intake port on the exterior surface of the retractor blade can connect via a channel passing through the blade to a smoke evacuation conduit positioned on the interior concave surface of the blade where it does not obstruct a surgeon's view of the surgical field during use. The device can provide for reduced interference with the surgeon's field of view and improved freedom of motion when operating through small skin incisions.

In some embodiments, a smoke removal device comprises a thin flat-bladed tissue retractor that incorporates a smoke evacuation conduit on a surface away from the surgeon's field of view. The device can effectively remove surgical smoke, while minimizing interference with the surgeon's field of view and maneuverability when operating through a small skin incision. In some embodiments, the evacuation conduit used to deliver negative pressure and to evacuate smoke is not placed on the exterior surface of the retractor blade (i.e., the surface that faces the surgeon during use), where even a small protrusion above the surface of the blade could significantly interfere with the surgeon's view through the narrowly separated incision edges. In certain embodiments, the smoke intake port on the retractor is placed in a location (e.g., not too near the distal or proximal ends of the retractor blade) such that it can effectively capture smoke without aspirating tissue from the surgical site. The device can be used to remove smoke produced from the coagulation and vaporization of tissues by an electrosurgical or laser apparatus, while minimizing the removal of body fluids, wound irrigation liquids, tissue debris, or other fluids.

In various embodiments, an apparatus for removing surgical smoke is provided. The device can comprise a tissue retractor and an evacuation conduit. The tissue retractor can comprise a retractor handle and/or a retractor blade. Any suitable handle may be used with the device disclosed herein. For example, the retractor blade can be attached to a handle or to a table mount, such as the handle 708 or the table mount 709 shown in FIG. 4. The retractor blade can comprise a proximal end, a distal end, a first interior surface, and a second exterior surface, wherein at least a portion of the first interior surface comprises a concave surface and at least a portion of the second exterior surface comprises a convex surface. In some embodiments, the retractor blade is attached to a retractor handle or other mounting device at the proximal end of the blade, e.g., the retractor shank 706 can be attached to the handle 708 via the connector 710 or to the table mount 709 via the connector 707, as shown in FIG. 4. In some embodiments, a smoke intake port on the exterior surface of the blade allows smoke to pass through a channel in the retractor blade near the distal end of the retractor blade, and from there the smoke passes into the evacuation conduit. In some embodiments, the evacuation conduit is affixed to the first interior surface of the retractor blade and operably connected to the smoke intake port on the exterior surface of the retractor blade, e.g., via a channel passing through the retractor blade from the interior to the exterior surface. In some embodiments, the evacuation conduit can be detachably connected to the retractor blade and/or handle. In some embodiments, the smoke intake port is positioned at least 10 mm from the distal end of the retractor blade and is flush with the exterior surface of the retractor blade. In some embodiments, the smoke intake port and retractor blade comprise attachment devices for attaching the evacuation conduit. These attachment devices can comprise at least one of a weld, loop, clip, snap, screw, or glue.

In some embodiments, a smoke removal system is provided. While not required for the devices and systems disclosed herein, in some embodiments, the system can comprise a negative pressure source, a tissue retractor, and an evacuation conduit. The tissue retractor can comprise a retractor handle and a retractor blade. The retractor blade can comprise a proximal end, a distal end, a first interior surface, and a second exterior surface, wherein at least a portion of the first interior surface comprises a concave surface and at least a portion of the second exterior surface comprises a convex surface. In some embodiments, the retractor blade is attached to the retractor handle at the proximal end of the blade. In some embodiments, a smoke intake port on the exterior surface of the retractor blade is operably linked to a channel (“smoke intake channel”) that passes through the retractor blade from the exterior to the interior surface near the distal end of the retractor blade. In some embodiments, the evacuation conduit is affixed to the first interior surface of the retractor blade and operably connected to the smoke intake port via the smoke intake channel, passing through the blade, and also is capable of being operably linked to the negative pressure source. In some embodiments, the evacuation conduit can be detachably connected to the retractor, the smoke intake port, and the negative pressure source. In some embodiments, the evacuation conduit has attachment devices for attaching to the retractor blade, the smoke intake port, and the negative pressure source. In certain embodiments, where the system optionally comprises a negative pressure source, the negative pressure source is a vacuum pump, a peristaltic pump, or a suction valve. In some embodiments, the negative pressure source can further comprise one or more filters to capture smoke withdrawn from the surgical site through the evacuation conduit.

In certain embodiments, a tissue retractor is provided. The retractor can comprise a retractor handle and a retractor blade. The retractor blade can comprise a proximal end, a distal end, a first interior surface, and a second exterior surface, wherein at least a portion of the first interior surface comprises a concave surface and at least a portion of the second exterior surface comprises a convex surface. In some embodiments, the retractor blade is attached to the retractor handle at the proximal end of the blade. In certain embodiments, the retractor blade is capable of pivoting on the handle and/or of being detached from the handle. In some embodiments, a smoke intake port on the exterior surface is operably linked to a smoke intake channel that passes through the retractor blade near the distal end of the retractor blade. In certain embodiments, the smoke intake port is positioned at least ½ inch, for example about 1 inch, from the distal end of the retractor blade and is flush with the exterior surface of the blade. In some embodiments, the retractor is capable of removing smoke generated during a surgical procedure when coupled to a negative pressure source, and the retractor blade can comprise attachment devices for attaching an evacuation conduit to the interior surface of the retractor blade without obscuring a surgeon's view of a surgical field.

In some embodiments, an evacuation conduit is provided, comprising a cannula having attachment devices for attaching the conduit to an interior surface of a retractor, to a smoke intake port, and to a negative pressure source, wherein the conduit is capable of delivering negative pressure from the negative pressure source to the smoke intake port. In some embodiments, the attachment devices on the evacuation conduit comprise at least one of welds, loops, clips, snaps, screws, or glues. In certain embodiments, the conduit is disposable.

In some embodiments, a method of removing smoke generated during a surgical procedure without obscuring a surgeon's view of a surgical field is provided. The method may comprise applying negative pressure at or near a surgical site through a surgical smoke removal system. The smoke removal system can comprise a negative pressure source, a tissue retractor, and an evacuation conduit. In some embodiments, the evacuation conduit is affixed to the first interior surface of the retractor blade and operably connected to the smoke intake port on the exterior surface of the retractor blade and to the negative pressure source. In some embodiments, the system is capable of removing smoke generated during a surgical procedure. In certain embodiments, the surgical smoke removal system is used as part of a procedure involving one or more small incisions, such as a face or neck lift. In some embodiments, the smoke removal system does not obscure the surgeon's field of view or freedom of motion during the surgical procedure.

### DESCRIPTION OF THE DRAWINGS

FIG. 1 shows a smoke removal retractor blade designed for attachment to various interchangeable handles or for attachment to a table-mounted articulated fixation arm, according to certain embodiments of the present disclosure.

FIGS. 2A-B are perspective views of a smoke removal retractor as a one-piece instrument, according to certain embodiments of the present disclosure. FIG. 2C is a cross-sectional view of FIG. 2A along line 509, while FIG. 2D is a cross-sectional view of FIG. 2B along line 510.

FIG. 3 shows an embodiment of a smoke removal retractor comprising a smoke evacuation cannula with fasteners for reversibly joining the cannula to a retractor.

FIG. 4 shows an embodiment of a smoke removal retractor with a quick connect-disconnect coupler for reversibly attaching to various handgrips or to a table-mounted immobilization apparatus.

FIG. 5A exemplifies a small incision surgery using a retractor, according to certain embodiments of the present disclosure. FIG. 5B shows the surgeon's view of a confined operating field using the retractor devices, according to certain embodiments of the present disclosure. An alternative arrangement for removing smoke in which the surgeon's view is obscured is shown in FIG. 5C.

### DESCRIPTION OF CERTAIN EXEMPLARY EMBODIMENTS

Reference will now be made in detail to certain exemplary embodiments according to the present disclosure, certain examples of which are illustrated in the accompanying drawings.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. To the extent publications and patents or patent applications incorporated by reference contradict the invention contained in the specification, the specification will supersede any contradictory material.

In this application, the use of the singular includes the plural unless specifically stated otherwise. Also in this application, the use of “or” means “and/or” unless stated otherwise. Furthermore, the use of the term “including,” as well as other forms, such as “includes” and “included,” are not limiting. Any range described here will be understood to include the endpoints and all values between the endpoints.

In this application, the words “conduit”, “cannula”, “catheter”, “channel”, and “tube” have the same meaning unless stated otherwise. Furthermore, the words plastic and polymeric have the same meaning. Similarly, “port”, “hole”, “porthole”, “vent”, “smoke intake port”, and “smoke intake port” have the same meaning and can be used interchangeably.

Disclosed herein are smoke removal devices and systems comprising a tissue retractor, a smoke evacuation conduit positioned along the interior surface of the retractor, and a smoke intake port positioned near the distal end of the retractor blade and attached to the evacuation conduit. Also disclosed herein are methods of using the devices and/or systems to remove surgical smoke.

In various embodiments, an apparatus for removing surgical smoke is provided. The apparatus can comprise a tissue retractor blade (with or without a handgrip) and an evacuation conduit running on or partially submerged within an interior surface of the retractor blade and operably linked to a smoke intake port on the exterior surface of the blade, e.g., approximately 1 inch from the distal tip of the blade. Negative pressure within the evacuation conduit can pull surgical smoke into the smoke intake port.

The retractor can have a variety of different shapes and configurations. In some embodiments, a retractor can comprise a retractor blade and an attached retractor handle. The retractor blade can be provided in a variety of shapes or sizes (e.g., rectangular, cylindrical, tapered, pointed, etc.). For example, a thin rounded retractor blade can be used for neck lifts or other small-incision procedures. The retractor blade can have a proximal end joined to the handle and a distal end that can be inserted into a tissue site. The distal end of the retractor blade can comprise points, claws, serrations, or flat ends. The retractor blade, or at least a portion of the retractor blade, can form a straight line with a curved distal end, form a smooth curve at the distal end, or provide a sharp angle at the distal end (e.g., a right angle). The retractor blade can have an interior surface and an exterior surface, wherein at least a portion of the interior surface comprises a concave surface running under the hooked, curved, or angled distal end of the retractor blade, and wherein at least a portion of the exterior surface runs along an opposite, convex side of the retractor blade.

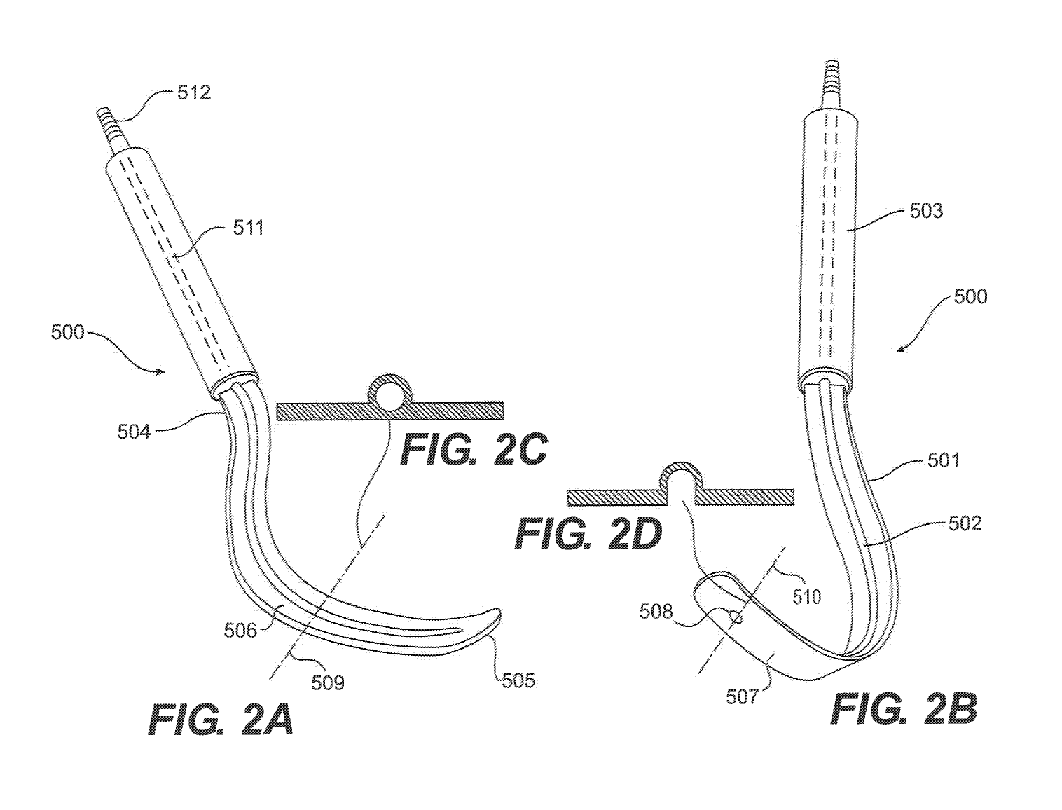

One suitable surgical retractor 500 according to certain embodiments of the present disclosure is shown in FIG. 2A-D, wherein the retractor 500 comprises a curved retractor blade 501 with a flat distal end 505.

In various embodiments, the retractor blade can be permanently attached to a handle or shaft, or it can be detachable. In some embodiments, retractor blades and handles are interchangeable and can be selected based on the components required for a given surgical procedure. The retractor blade and/or handle can be disposable and/or reusable (e.g., suitable for autoclave sterilization) and can be made from a variety of suitable materials (e.g., polymeric or metallic materials).

The retractor can be designed for hand-held use or for use with a mechanical fixation element on a surgical table (e.g., a table-mounted articulated fixation arm).

In certain embodiments, the smoke-removing retractor is comprised of a single flat rigid retractor blade, as opposed to a twin-bladed (i.e., bi-valved) speculum, or a cylindrical speculum. In some embodiments, the retractor blade can vary in width (e.g., about ½″, ⅝″, ¾″, ⅞″, or 1″), length, overall configuration (e.g., C-shape, S-shape, L-shape), tip design (e.g. square, rounded, pointed, curled), and/or thickness. In some embodiments, the thickness of the blade should be minimized, while maintaining the necessary rigidity and fracture resistance for surgical use. For example, a blade could comprise a thickness of about 1/16″—not including the increased thickness produced by the smoke evacuation conduit running along the blade.

In various embodiments, a retractor and/or retractor blade has an exterior and an interior surface. As used herein, the “interior surface” of the retractor is the surface that comes into direct contact with the tissue being lifted by the retractor, and is not visible to the surgeon during use. The “exterior surface” of the retractor is the surface opposite to the interior surface. For example, FIG. 2A-D shows a retractor 500 comprising a retractor blade 501 having an interior surface 506 and an exterior surface 507. The evacuation conduit 502 runs along the interior surface 506 and is operably linked to the smoke intake port 508 at the distal end 505 of the retractor blade 501.

In some embodiments, the retractor blade can be permanently joined to a handgrip (e.g., a handle) with the smoke evacuation conduit partially or completely attached to one side of the handle. Alternatively, the conduit can run partially or completely within the handle, or can terminate without joining the handle at all. In other embodiments, the retractor blade can reversibly attach to interchangeable handles of various sizes and shapes, e.g., using a quick connect-disconnect coupler. In some embodiments, the retractor blade can reversibly attach to an articulated-arm, e.g., a cable-tightened apparatus that securely mounts to a rail or other surface on an operating table for a rigid positioning of the retractor.

For example, FIG. 2A-D illustrates an embodiment of the smoke removal retractor 500 with a retractor blade, smoke intake port, evacuation conduit, and handle integrated into a one-piece instrument (e.g., a disposable plastic instrument made using, e.g., an injection-molding process). In some embodiments, the one-piece instrument avoids the inconvenience of assembling component parts prior to use and, if the instrument is disposable, avoids the need to clean and sterilize a narrow cannula. The exemplary instrument shown in FIG. 2A-D comprises a retractor blade 501, a smoke evacuation conduit 502, and a handle 503. The retractor blade has a proximal end 504, and a distal tip 505, a first interior surface 506, and a second exterior surface 507, wherein at least a portion of the first interior surface 506 comprises a concave surface and at least a portion of the second exterior surface 507 comprises a convex surface. The retractor blade can incorporate a smoke intake port 508 located proximal to the distal tip of the retractor blade. In some embodiments, to reduce the combined thickness of the blade and conduit, the conduit can be partially submerged below the first interior surface of the blade (as shown in cross-section along line 509 of FIG. 2C) while still in communication with the smoke intake port (as shown in cross-section along line 510 of FIG. 2D). The evacuation conduit can run through the handle 503 to merge with a tapered connector 512.

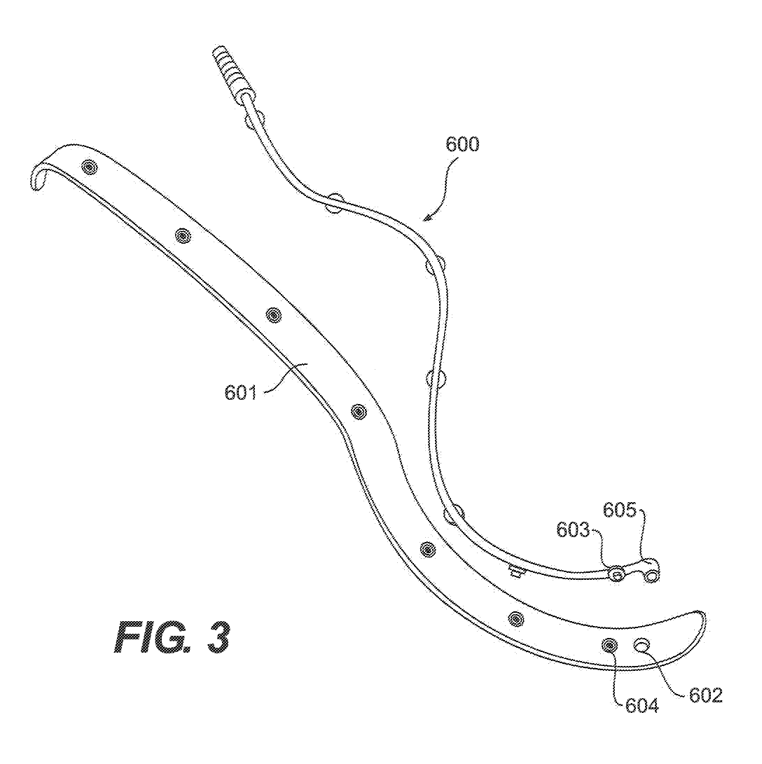

In another example, as shown in FIG. 3, a single-use flexible smoke evacuation cannula 600 can be affixed to the interior surface of a Deaver-style retractor 601 that contains a distal smoke intake port 602. Male snap fasteners on the catheter 603 can attach to female snap fasteners 604 on the retractor. A firm hollow elbow plug 605 can be placed at the distal end of the cannula and can connect the cannula to the smoke intake port 602. In some embodiments, other types of fasteners can be used to attach the cannula. In some embodiments, the retractor can comprise blades and handles of different shapes, widths, and lengths.

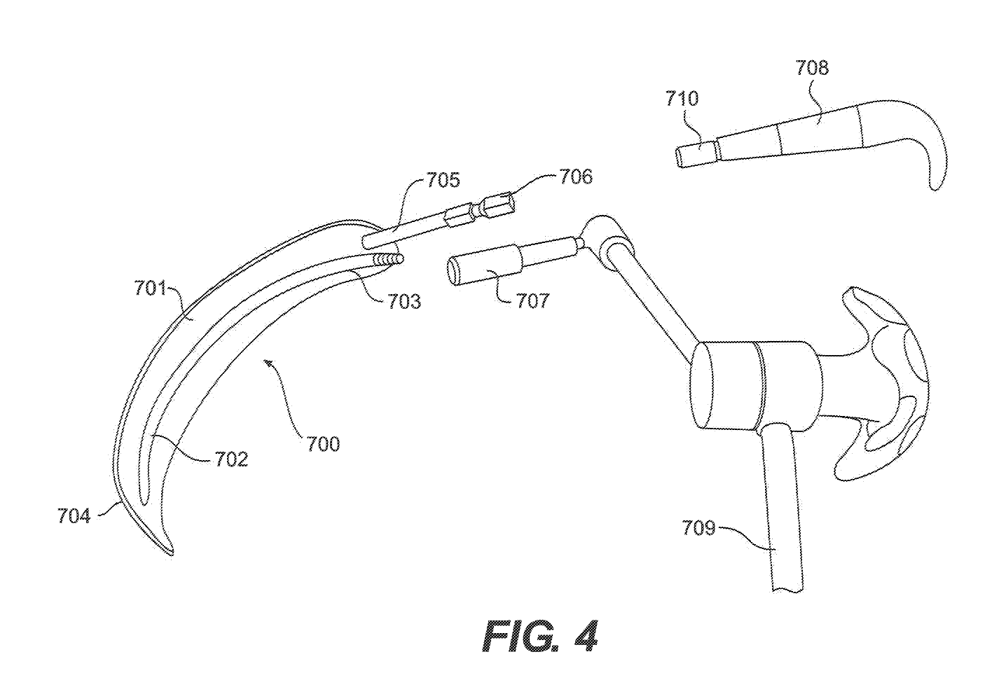

In yet another example, FIG. 4 shows a smoke removal retractor 700 comprising a retractor blade 701, smoke evacuation conduit 702 with a tapered tubing connector 703, and a smoke intake port located near the tip of the blade 701. The proximal portion of the retractor 705 is fitted with an example of a male quick connect-disconnect fitting 706 for reversibly attaching to a female quick connect-disconnect fitting 707 on a variety of interchangeable handles, or for reversibly attaching to a table-mounted, cable-tightened immobilization apparatus 709. In some embodiments, a retractor blade, smoke evacuation conduit, and quick connect coupler can be supplied as a one-piece instrument (disposable or reusable), or as multi-piece unit.

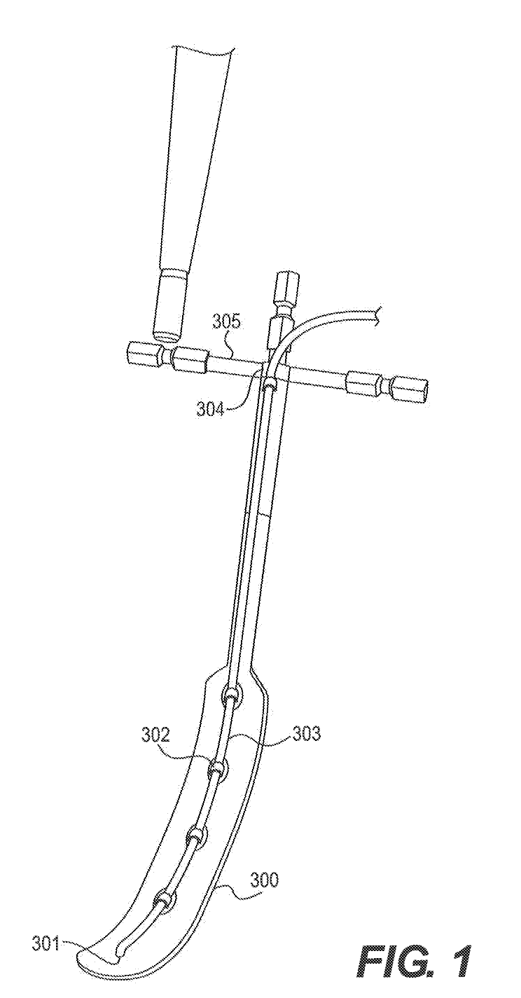

In various embodiments, a smoke removal apparatus comprises an evacuation conduit running along the interior surface of the retractor. The conduit can comprise a cannula running at least the length of the retractor and extending from the proximal end of the retractor to a negative pressure source. For example, FIG. 2A-D shows an evacuation conduit 502 on the interior surface 506 of retractor blade 501, and the smoke intake port 508 on the exterior surface 507 of the blade 501, while FIG. 1 shows a conduit 303 on the interior surface of retractor 300.

In some embodiments, the smoke evacuation conduit can be permanently or reversibly cast, welded, and/or affixed onto the interior surface of the retractor blade, with the distal end of the conduit permanently or reversibly connected to the smoke intake port. In certain embodiments, the conduit can be partially submerged below the interior surface of the retractor blade such that it produces a reduced profile hump on the interior surface, but does not produce a visible hump or spine on the exterior surface of the blade.

The evacuation conduit can comprise any material suitable for channeling negative pressure to a surgical site. In some embodiments, the conduit comprises a hollow polymeric or metallic material. In some embodiments, the conduit is rigid. In other embodiments, the conduit is flexible (e.g., capable of being used with retractor blades of different shapes, sizes, and/or properties). In certain embodiments, the conduit has a cross-sectional diameter of about 1 mm-1 cm (e.g., about 1, 5, 10, 20, 50, 100, 500, or 1000 mm, or any diameter in between). The evacuation conduit can be disposable or reusable and can be permanently attached to the retractor or can be detachable. In certain embodiments, the evacuation conduit can be attached to the retractor using welded metal strips, snap fasteners, hooks, pins, screws, cleats, rivets, loops, clips, adhesives, glues, or other attachment methods. For example, FIG. 3 shows an evacuation conduit 600 attached to a retractor by snaps 603 and 604, while FIG. 1 shows a conduit 303 attached to retractor 300 by loops 302.

In various embodiments, the smoke evacuation conduit can be flexible, rigid, or a combination of the two. Its cross-sectional shape can be any suitable shape for removing smoke, e.g., round, oval, square, or rectangular. The cross-section can be provided in a variety of sizes, as long as the internal dimensions permit enough flow of air to extract surgical smoke, and as long as the external dimensions do not compromise the low-profile of the instrument. For example, a round thin-walled cannula with about ⅛″ inner diameter and about 3/16″ outer diameter could be used. The length of the evacuation cannula can vary according to the overall design of the smoke removal apparatus. In some embodiments, the proximal end of the cannula can incorporate a rigid tapered tubing adapter (smooth or barbed) that can interface directly or indirectly with a wall-mounted or free-standing negative pressure device. In some embodiments, the negative pressure device is one dedicated to smoke evacuation, with a foot-controlled on/off switch operated by the surgeon or the surgical assistant. In some embodiments, the tapered tubing adapter can have an inner diameter that is larger than the outer diameter of a needle adapter on a plastic syringe, such that the cannula can be flushed out using the syringe if the cannula becomes obstructed with aspirated tissue debris during surgery.

In various embodiments, a smoke removal system includes a smoke intake port located near the distal end of the retractor blade (the end of the blade furthest from the retractor handle). For example, FIG. 2A-D shows a smoke intake port 508 at the distal end 505 of the retractor blade 501, and FIG. 1 shows a distal end 301 of the smoke evacuation conduit 303 that can be communicatively connected to a smoke intake port on the exterior surface of the blade in order to channel smoke away from the surgical site. In some embodiments, the smoke intake port is operably linked to a smoke intake channel that passes through the retractor blade from the exterior surface of the blade to the interior surface and provides a channel for surgical smoke to pass through the retractor and into the evacuation conduit. In some embodiments, the smoke intake port is flush with the exterior surface of the blade to avoid interference with vision and is positioned approximately one inch from the distal tip of the blade to avoid obstruction from tissue aspiration at or near the port. In some embodiments, the smoke intake port is operably linked contact with the evacuation conduit (e.g., a permanent connection such as a weld or a detachable connection such as a snap, screw, glue, or other connection method). In some embodiments, the negative pressure provided by the evacuation conduit pulls surgical smoke through the smoke intake port and into the attached evacuation conduit.

In some embodiments, the smoke intake port can vary in size and shape (e.g. round, oval, square, or rectangular) to permit an unimpeded flow of aspirated smoke into the smoke evacuation conduit. In some embodiments, the port can be located approximately 1 inch (e.g., about 25 mm) from the distal tip of the retractor blade, or about 0.5 inches from the distal tip. In some embodiments the smoke intake port is operably linked to a smoke intake channel that penetrates the blade from front-to-back and can connect to a smoke evacuation conduit that runs down the interior surface in such a way that a near air-tight seal is created that prevents an unwanted suction leak around the cannula to the interior surface of the retractor blade. In some embodiments, a smoke evacuation cannula can be distally fitted with a hollow 90° male elbow plug that tightly but reversibly connects with a female socket comprising the smoke intake port.

In some embodiments, each component of the smoke removal system can be disposable and/or reusable (e.g., suitable for autoclave sterilization), as well as combinations of components. For example, the retractor blade can be autoclavable while the evacuation conduit is disposable and is detached from the blade after each use. In another example, both the retractor blade and the evacuation conduit are disposable and are detached from an autoclavable retractor handle after use.

In some embodiments, the retractor blade, the smoke evacuation cannula, and/or the handle can be made of a disposable plastic material. In some embodiments, the retractor blade, the smoke evacuation cannula, and/or the handle can be made of a sterilizable (e.g. autoclavable) material, such as a metal material (e.g., stainless steel, aluminum, or titanium). In some embodiments, one benefit of a disposable evacuation conduit or other component is that it precludes the difficulties inherently associated with cleaning and sterilizing a narrow lumen cannula.

In various embodiments, the evacuation conduit is capable of delivering negative pressure to a surgical site in order to remove surgical smoke. In certain embodiments, the negative pressure is provided by a negative pressure source that is connected to the evacuation conduit. The negative pressure source can be any device capable of providing negative pressure (e.g., a vacuum pump, peristaltic pump, etc.). In certain embodiments, the negative pressure source can comprise one or more filters that capture and collect the smoke withdrawn from the surgical site via the evacuation conduit. In some embodiments, the conduit can be configured to provide a sealed (e.g., airtight) attachment to a negative pressure device and/or to a suction valve installed in the wall of an operating theater. For example, FIG. 3 shows an evacuation conduit 600 having an attachment for a negative pressure device and an attachment 605 for attaching the conduit 600 to a smoke intake port 602.

In various embodiments, negative pressure from the evacuation conduit pulls surgical smoke through the smoke intake port and down the evacuation conduit, removing the smoke from the surgical site. In some embodiments, the smoke intake port is positioned near the distal end of the retractor blade, but not at the most distal point or tip of the retractor blade (e.g., about 0.5, 0.6, 0.7, 0.8, 0.9, 1.0, 1.5, 2.0, or 2.5 inches from the distal tip of the blade, or any distance in between). In certain embodiments, positioning the smoke intake port near but not at the distal tip improves the efficiency of smoke removal by optimizing the smoke intake port's location within the center of the smoke plume while reducing the risk that the port will become obstructed by aspirated tissue (as compared to a smoke intake port positioned at or adjacent to the distal end of the retractor blade).

The devices and systems disclosed herein can be packaged and provided as a kit. In some embodiments, the kit comprises a smoke removal retractor and instructions for using the retractor. In some embodiments, the kit comprises an evacuation conduit that is permanently attached to the retractor. In other embodiments, the evacuation conduit is detachable from the retractor and can be provided either pre-attached or disassembled. In some embodiments, the kit can include one or more (e.g., 1, 2, 3, 4, 5, or more) retractors and/or evacuation conduits. In some embodiments, the kit can further comprise a negative pressure device and, optionally, instructions for attaching the negative pressure device to the smoke removal retractor. In certain embodiments, the components of the kit can be provided in sterile form.

In various embodiments, the smoke removal devices and systems described above can be used during a surgical procedure to remove smoke. For example, after normal surgical preparation (e.g., draping and sterilization), one or more smoke removal retractors can be mounted on the table (or held by hand) in the desired location(s), an incision can be made, and the appropriate tissue tented using the smoke-removing retractor(s). The negative pressure source can be activated to apply negative pressure at or near the surgical site. During the surgical procedure, standard cauterization techniques (e.g., using an electrosurgical tool) can be applied to reduce bleeding, as necessary. Likewise, standard laser techniques can also be applied as appropriate in a given procedure. Smoke generated during the procedure (e.g., from the cauterization process or the surgical laser use) is withdrawn through the smoke intake port and down the evacuation conduit. As the evacuation conduit is on the interior surface of the retractor, the evacuation conduit is not visible to the surgeon and do not obstruct the surgeon's line of sight or freedom of motion within the surgical site (e.g., it does not obscure a portion of the incision, as it would if positioned on the exterior surface of the retractor).

In certain embodiments, the smoke removal devices and systems described above are used as part of a procedure involving small surgical incisions, e.g., incisions of about 0.5-2.0 inches, such as those used in laparoscopic surgery (e.g., minimally invasive abdominal procedures), plastic and reconstructive surgery (e.g. breast augmentation, lumpectomy, and face, neck, or chin augmentation), or any other procedure in which small incisions and tissue retraction are required. In these small incision surgeries, there is a greater need to avoid occlusion of the surgeon's field of view and to retain the surgeon's freedom of motion. Even a small cannula passing along the exterior surface of a retractor or otherwise positioned near a small incision to remove surgical smoke can obscure a significant portion of the surgical field during these small incision surgeries. The retractors of the present disclosure address this problem by providing an evacuation conduit on the internal surface of a retractor. The conduits are thereby hidden by the retractor and do not intrude into the surgical field. Accordingly, the present devices and systems address the problem of surgical field obstruction while still allowing for efficient removal of surgical smoke. Furthermore, by providing a smoke intake port near but not at the distal end of the retractor, the efficiency of smoke removal can be improved while reducing the risk that the port will become obstructed by aspirated tissue.

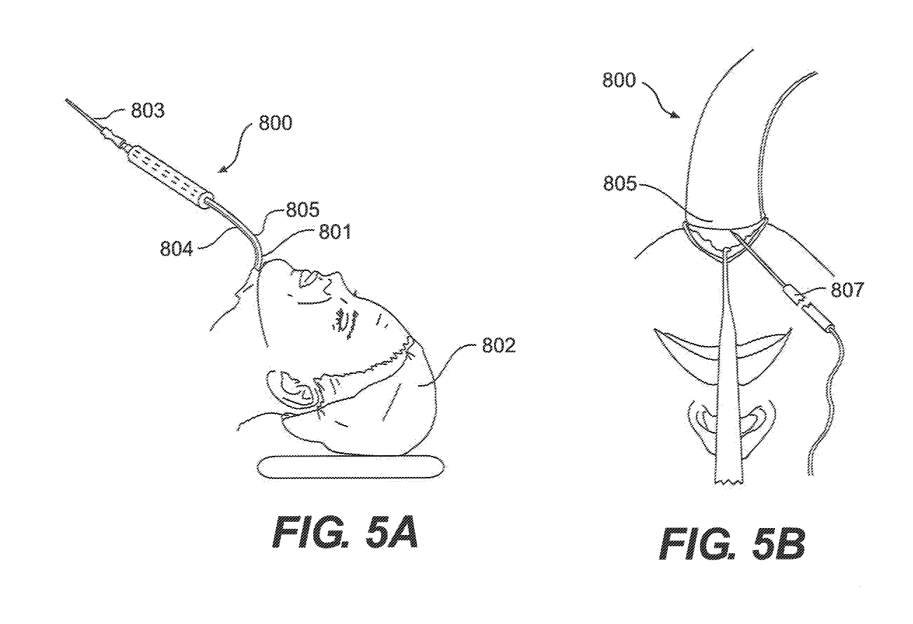

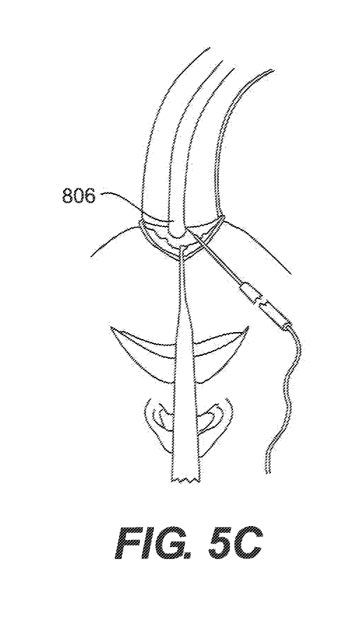

In some embodiments, the devices described above can be used in a small incision surgical procedure. In some embodiments, the devices and systems comprise a retractor having a flat exterior surface, a smoke intake port positioned flush with the exterior surface of the retractor blade about 0.5-1.5 inches from the distal tip, and a smoke evacuation conduit positioned on the interior surface of the blade where it does not obscure the field of view. For example, the devices can be used as shown in FIG. 5A and FIG. 5B, which illustrates an exemplary plastic surgery “neck lift”, which is typically performed through a small transverse skin incision placed just under the chin. The patient 802 lies supine on the operating table. The surgeon (not shown) stands or sits at the head of the table, optionally wearing a surgical headlight to illuminate the interior of the subcutaneous pocket. With the retractor connected by tubing 803 to a negative pressure source, and with its blade holding the incision open and tenting up the skin flap, a long electrosurgical “pencil” 807 can be used to dissect and re-shape the subcutaneous tissues of the neck while maintaining hemostasis. With the retractor's evacuation conduit concealed on the interior surface 804 of the retractor blade, there is no protrusion on the exterior surface of the blade 805 to interfere with the surgeon's view into the operative pocket. As shown in FIG. 5B, the smoke intake port located on the exterior surface of the retractor blade is unseen because the distal portion of the blade on which the port resides is out of sight. For comparison, FIG. 5C is the same view but without using an exhaust cannula placed under the interior surface of the retractor blade, producing a visual obstruction to the surgeon's view within the confines of the small incision opening.

The preceding examples are intended to illustrate and in no way limit the present disclosure. Other embodiments of the disclosed systems, devices, and methods will be apparent to those skilled in the art from consideration of the specification and practice of the systems, devices and methods disclosed herein.

What is claimed is:

1. An apparatus for removing surgical smoke, comprising:
a tissue retractor, comprising:
a retractor blade comprising a proximal end, a distal end, a first interior surface, and a second exterior surface, wherein at least a portion of the first interior surface of the retractor blade comprises a concave surface and at least a portion of the second exterior surface comprises a convex surface; and
a smoke intake port on the exterior surface that is operably linked to a smoke intake channel passing through the retractor blade near the distal end of the retractor blade; and
an evacuation conduit affixed to the first interior surface of the retractor blade and operably connected to the smoke intake port,
wherein the system is capable of removing smoke generated during a surgical procedure.

2. The apparatus of claim 1, wherein the evacuation conduit can be detachably connected to the retractor.

3. The apparatus of claim 1, wherein the retractor is autoclavable.

4. The apparatus of claim 1, wherein the smoke intake port is positioned at least about 0.5 inches from the distal end of the retractor blade and is flush with the exterior surface of the retractor blade.

5. The apparatus of claim 1, wherein the smoke intake port comprises an attachment device for the evacuation conduit.

6. The apparatus of claim 5, wherein the attachment device comprises at least one of a weld, loop, clip, snap, or screw.

7. The apparatus of claim 1, wherein the retractor comprises at least one attachment device for the evacuation conduit on the interior surface of the retractor blade.

8. The apparatus of claim 7, wherein the at least one attachment device comprises at least one of a weld, loop, clip, snap, or screw.

9. The apparatus of claim 1, wherein the evacuation conduit comprises a rigid metallic cannula.

10. The apparatus of claim 1, wherein the evacuation conduit comprises a flexible polymeric cannula.

11. The apparatus of claim 1, wherein the evacuation conduit comprises attachment devices for attaching the conduit to the interior surface of the retractor blade and to the smoke intake port.

12. The apparatus of claim 11, wherein the attachment devices comprise at least one of welds, loops, clips, snaps, or screws.

13. A kit comprising the apparatus of any one of claims 1-12 and instructions for using the apparatus.

14. A smoke removal system comprising:
a negative pressure source;
a tissue retractor, comprising:
a retractor blade comprising a proximal end, a distal end, a first interior surface, and a second exterior surface, wherein at least a portion of the first interior surface of the retractor blade comprises a concave surface and at least a portion of the second exterior surface comprises a convex surface; and
a smoke intake port on the exterior surface that is operably linked to a smoke intake channel passing through the retractor blade near the distal end of the retractor blade; and
an evacuation conduit affixed to the first interior surface of the retractor blade and operably connected to the smoke intake port and to the negative pressure source,
wherein the system is capable of removing smoke generated during a surgical procedure.

15. The system of claim 14, wherein the smoke intake port is positioned at least about 0.5 inches from the distal end of the retractor blade and is flush with the exterior surface of the blade.

16. The system of claim 14, wherein the smoke intake port and retractor blade comprise attachment devices for the evacuation conduit.

17. The system of claim 16, wherein the attachment devices comprise at least one of welds, loops, clips, snaps, or screws.

18. The system of claim 14, wherein the evacuation conduit comprises attachment devices for attaching the conduit to the interior surface of the retractor blade, to the smoke intake port, and to the negative pressure source.

19. The system of claim 18, wherein the attachment devices comprise at least one of welds, loops, clips, snaps, or screws.

20. The system of claim 14, wherein the negative pressure source is a vacuum pump, a peristaltic pump, or a suction valve.

21. The system of claim 20, wherein the negative pressure source further comprises one or more filters to capture smoke withdrawn from the surgical site through the evacuation conduit.

22. An evacuation conduit comprising a cannula having attachment devices for attaching the conduit to an interior surface of a retractor, to a smoke intake port, and to a negative pressure source, wherein the conduit is capable of delivering negative pressure from the negative pressure source to the smoke intake port.

23. The conduit of claim 22, wherein the attachment devices comprise at least one of welds, loops, clips, snaps, or screws.

24. The conduit of claim 22, wherein the conduit is disposable.

25. The conduit of claim 22, wherein the conduit is autoclavable.

26. The conduit of claim 22, wherein the conduit comprises a cannula having a cross-sectional dimension of about 1 mm-1 cm.

27. The conduit of claim 22, wherein the conduit comprises a rigid cannula.

28. The conduit of claim 22, wherein the conduit comprises a flexible polymeric cannula.

29. A tissue retractor comprising:
a retractor blade comprising a proximal end, a distal end, a first interior surface, and a second exterior surface, wherein at least a portion of the first interior surface of the retractor blade comprises a concave surface and at least a portion of the second exterior surface comprises a convex surface; and
a smoke intake port on the exterior surface that is operably linked to a smoke intake channel passing through the retractor blade near the distal end of the retractor blade; and
wherein the retractor is capable of removing smoke generated during a surgical procedure when coupled to a negative pressure source, and wherein the retractor blade comprises attachment devices for attaching an evacuation conduit to the interior surface of the retractor blade without obscuring a surgeon's view of a surgical field.

30. The retractor of claim 29, wherein the retractor is disposable.

31. The retractor of claim 29, wherein the retractor is autoclavable.

32. The retractor of claim 29, wherein the retractor blade is permanently or reversibly attached to a retractor handle.

33. The retractor of claim 32, wherein the retractor blade is capable of pivoting on the handle and is capable of being locked in position.

34. The retractor of claim 29, wherein the smoke intake port is positioned at least about 0.5 inches from the distal end of the retractor blade and is flush with the exterior surface of the blade.

35. The retractor of claim 29, wherein the smoke intake port and retractor comprise attachment device for the evacuation conduit.

36. The retractor of claim 35, wherein the attachment devices comprise at least one of welds, loops, clips, snaps, or screws.

37. A method of removing smoke generated during a surgical procedure without obscuring a surgeon's view of a surgical field, comprising applying negative pressure at or near a surgical site through a surgical smoke removal system, wherein the system comprises
a negative pressure source;
a tissue retractor, comprising:
a retractor blade comprising a proximal end, a distal end, a first interior surface, and a second exterior surface, wherein at least a portion of the first interior surface of the retractor blade comprises a concave surface and at least a portion of the second exterior surface comprises a convex surface; and
a smoke intake port on the exterior surface that is operably linked to a smoke intake channel passing through the retractor blade near the distal end of the retractor blade; and
an evacuation conduit removably affixed to the first interior surface of the retractor blade and operably connected to the smoke intake port and the negative pressure source,
wherein the system is capable of removing smoke generated during a surgical procedure.

38. The method of claim 37, wherein the surgical smoke removal system is used as part of a procedure involving one or more small surgical incisions.

39. The method of claim 38, wherein the surgical smoke removal system does not obscure a surgeon's field of view or freedom of motion during the surgical procedure.

40. The method of claim 38, wherein the procedure is a face or neck lift.
